(19) United States
(12) Patent Application Publication
(10) Pub. No.: US 2020/0298061 A1
(43) Pub. Date: Sep. 24, 2020

(54) COMPUTER IMPLEMENTED METHOD OF DETERMINING ATHLETIC APTITUDE

(71) Applicant: ROOKIE ME IP PTY LTD, Lysterfield (AU)

(72) Inventors: Marco ROSANO, Lysterfield (AU); Adham DIMACHKI, Lysterfield (AU)

(21) Appl. No.: 16/841,916

(22) Filed: Apr. 7, 2020

Related U.S. Application Data
(63) Continuation of application No. 15/024,978, filed on Mar. 25, 2016, now abandoned.

(30) Foreign Application Priority Data
Sep. 27, 2013 (AU) 2013903744

(51) Int. Cl. A63B 24/00; G06F 16/9535; A63B 71/06

(52) U.S. Cl. CPC A63B 24/0062; G06F 16/9535; A63B 2024/0068; A61B 2503/10; A63B 2024/0065; A63B 71/0616

(57) ABSTRACT

There is disclosed a computer implemented method of determining athletic aptitude, The method may comprise: (a) determining a plurality of metrics in relation to a sportsperson, (b) entering the plurality of metrics into a database; (c) forming a profile of the sportsperson comprising the plurality of metrics; and, (d) comparing the sportsperson profile with the reference profile to provide a sportsperson comparison output, wherein the sportsperson comparison output is depicted on a graphic user interface interconnected to the database.

### FIELD OF THE INVENTION

The present invention relates, in part, to a computer implemented method. In a particular aspect, the present invention relates to a computer implemented method of determining athletic aptitude.

The invention has initially been developed for use in relation to elite (or near-elite) sportspeople whether in athletics or other sports and a computer implemented method of determining athletic aptitude is described with reference to this application. However, it will be appreciated that the invention is not limited to this particular field of use and has many potential applications.

### BACKGROUND OF THE INVENTION

Determining a prospective sportsperson's athletic aptitude is now a necessary requirement prior to, for example, offering a contract or position in a team sport at an elite or professional level. Increasingly this is also the case in sub-elite or amateur level sports. Every sport, around the world, typically has a set of testing criteria that it uses to gauge a sportspersons ability or athletic aptitude. All the way from grass roots to the elite level, sporting clubs, coaches and organisations test sportspeople to try and make accurate assessments of, for example, their aerobic capacity, agility, speed and/or strength. All of this is done with the aim of trying to better understand an individual's ability or athletic aptitude either in general or for a particular sport or discipline.

An example of standard testing criteria exists at the Australian Football League (AFL) Draft Combine/Camp. Prospective AFL players are put through the following tests and graded against one another:
Vertical Jump
Running Vertical Jump
Agility Run
20 metre Sprint
Repeat Sprint
Shuttle Run/Beep Test
3 kilometre Time Trial (running)
Kicking Efficiency
Clean Hands
Goal Kicking Test

However, currently these metrics are typically used only to check if they fall within pre-determined qualifying or acceptable ranges. Therefore, if a prospective AFL player falls below or outside one or more of the pre-determined ranges, they are typically considered not suitable. There is very little objective analysis relative to each individual's range of metrics or relative to other important considerations. This may also make it difficult for a prospective professional sportsperson to know, before the testing, how to specifically improve their fitness and skills and improve prospects of selection.

Prospective professional sportspeople may not know how potential peers in different locations and/or clubs perform in relation to the standard testing criteria for their sport. This applies particularly to sportspeople located in remote or rural areas. Also, prospective professional sportspeople may have as yet unidentified potential athletic aptitude in relation to a sport, particular discipline or playing position of which they are unaware. Despite advances, the process of identifying such potential in an individual still involves a considerable amount of ‘luck’ in that, for example, the individual has access to or otherwise becomes involved in a talent identification program in relation to sport(s) for which they have underlying potential athletic aptitude.

Further, prospective professional sportspeople in individual or non-team sports, for example, may perform the majority of their training alone. Consistently training alone requires considerable motivation and can be difficult to sustain where insight as to progress relative to peers might only be gained in competition, which may occur only infrequently.

It is an object of the present invention to substantially overcome or at least ameliorate one or more of the disadvantages of the prior art, or to at least provide an alternative.

### SUMMARY OF THE INVENTION

According to an aspect, there is provided a computer implemented method of determining athletic aptitude. The method may comprise: (a) determining a plurality of metrics in relation to a sportsperson; (b) entering the plurality of metrics into a database; (c) forming a profile of the sportsperson comprising the plurality of metrics; and, (d) comparing the sportsperson profile with a reference profile to provide a sportsperson comparison output, wherein the sportsperson comparison output is depicted on a graphic user interface interconnected to the database.

The plurality of metrics may relate to a team sport and the reference profile may relate to a position or a sub-set of positions in the team sport. The plurality of metrics may relate to Australian rules football. The plurality of metrics may includes two or more of:
(a) Vertical Jump;
(b) Running Vertical Jump;
(c) Agility Run;
(d) 20 metre Sprint;
(e) Repeat Sprint;
(f) Shuttle Run/Beep Test;
(g) 3 kilometre Time Trial (running);
(h) Kicking Efficiency;
(i) Clean Hands;
(j) Goal Kicking Test.

The reference profile may be formed from a previously determined set of metrics in relation to the sportsperson. The reference profile may be formed from another sportsperson profile included in the database. The reference profile may be formed from a set of metrics taken from two or more other sportsperson profiles included in the database. The reference profile may be formed in real-time as the metrics are received over a network by the database and the other sportsperson profile(s) are formed. The reference profile may be formed from a set of pre-determined metrics forming a virtual best sportsperson.

The reference profile may further comprise one or more sportsperson profile parameter(s). A sportsperson profile parameter may be age, weight or height A sportsperson profile parameter may be a particular sport. A sportsperson profile parameter may be a position or sub-set of positions played in a particular sport. The particular sport may be Australian rules football or a rugby code. A sportsperson profile parameter(s) may be a team of the sportsperson in the sportsperson profile.

A user may select one or more sportsperson profile parameters via the graphic user interface and a reference determinator may determines the reference profile to be compared to the sportsperson profile. The reference determinator may determine the reference profile to be from another sportsperson profile included in the database matched to the selected one or more player profile parameters. The reference determinator may determine the reference profile to be formed from a set of metrics taken from two or more other sportsperson profiles included in the database matched to the selected one or more player profile parameters.

Other aspects are also disclosed.

### BRIEF DESCRIPTION OF THE DRAWINGS

Notwithstanding any other forms which may fall within the scope of the present invention, a preferred embodiment preferred embodiments of the invention will now be described, by way of example, with reference to the accompanying drawings in which:

FIG. 1 is a diagrammatic view of a method of collecting and comparing metrics in a computer implemented method of determining athletic aptitude in accordance with a preferred embodiment of the present invention; and

FIG. 2 is diagrammatic flow diagram of collecting and comparing metrics of a computer implemented method of FIG. 1;

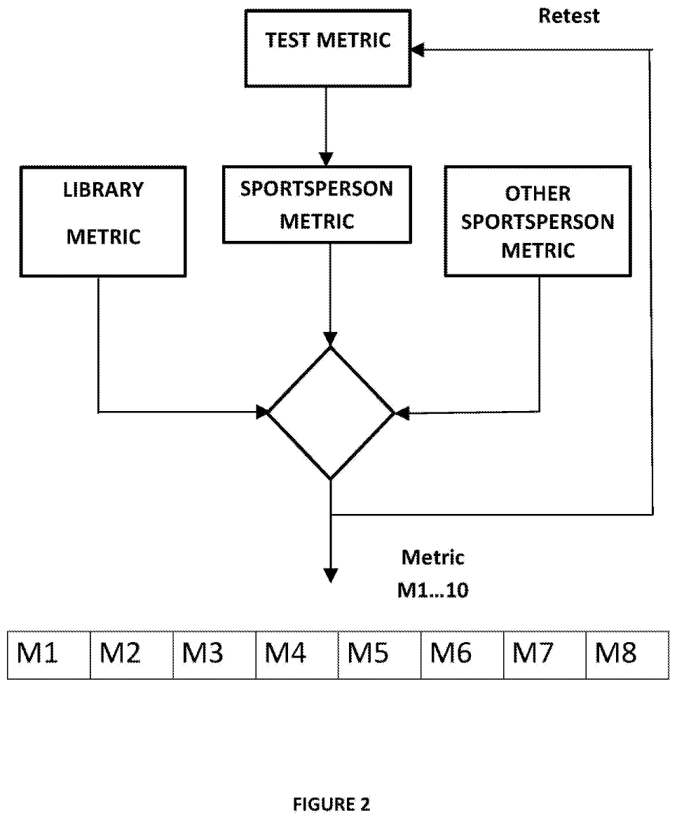

FIG. 3 is a diagrammatic block diagram of a computer and telecommunication network in use providing a computer implemented method of determining athletic aptitude in accordance with a preferred embodiment of the present invention;

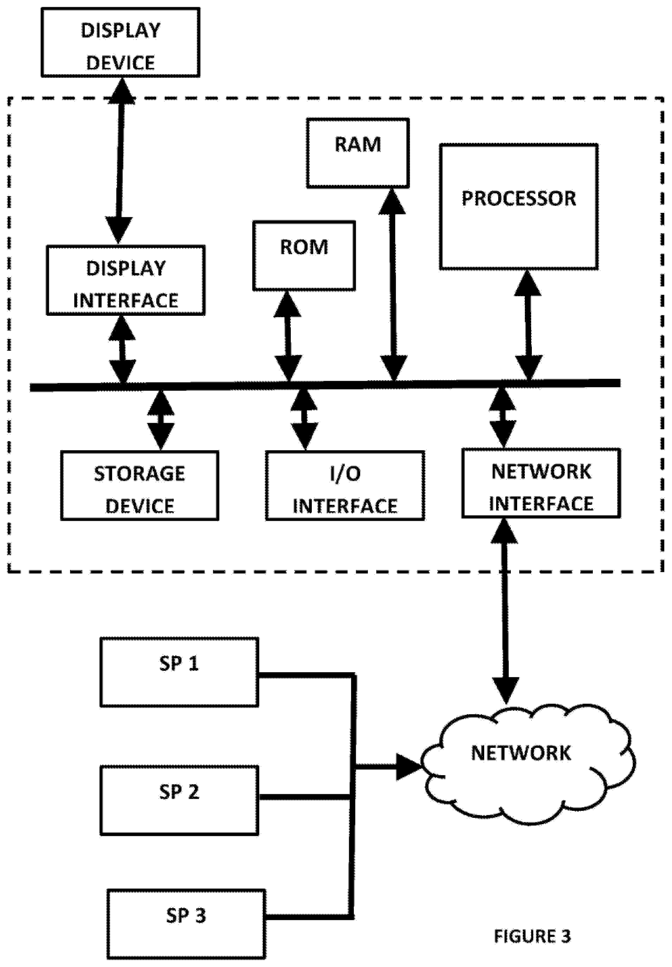

FIG. 4 is a diagrammatic flow diagram of a Compare Me profile compare in a computer implemented method of determining athletic aptitude in accordance with a preferred embodiment of the present invention.

### DESCRIPTION OF PREFERRED EMBODIMENTS

It should be noted in the following description that like or the same reference numerals in different embodiments denote the same or similar features.

Referring to the drawings there is shown, in part, a computer implemented method of determining athletic aptitude.

Preferred embodiments use web communication to help users determine and manage their sporting ability to reach their potential. Users have the ability to create profiles within a particular sport which may then allow them to compare themselves (using, for example, the “Compare Me” function detailed below) to other users on the system within the same sport or other sub-sets of users. In doing this, users may be provided with information and insights into where their ability sits in relation to their direct competition (within their desired sport) and know who their competition is.

Preferred embodiments of the present invention have metrics as a starting point. The metrics may be general and/or sport specific. An example of sport specific testing criteria exists at the AFL Draft Combine/Camp for the sport of Australian rules football. Prospective AFL players are put through the following tests and graded against one another:
Vertical Jump
Running Vertical Jump
Agility Run
20 metre Sprint
Repeat Sprint
Shuttle Run/Beep Test
3 kilometre Time Trial (running)
Kicking Efficiency
Clean Hands
Goal Kicking Test

The accurate input of metrics by users improves the utility of the system.

As mentioned above, every sport typically has a set of standard tests and subsequent metrics. These metrics may be entirely sport specific or they may include sport specific metrics and more general metrics. For example, in the AFL example detailed above, the Goal Kicking Test is sport specific, however the 20 metre Sprint is a more general metric, although it is specifically relevant to athletic aptitude for Australian rules football. Where the particular sport has a set of standard tests and subsequent metrics, historical records for each metric may be stored from previously tested athletes along with personal information such as a user's name, email address, sport, playing position. A user's metrics PP1, PP2 . . . may also be recorded and stored for further and future analysis.

### A) Metric Compare

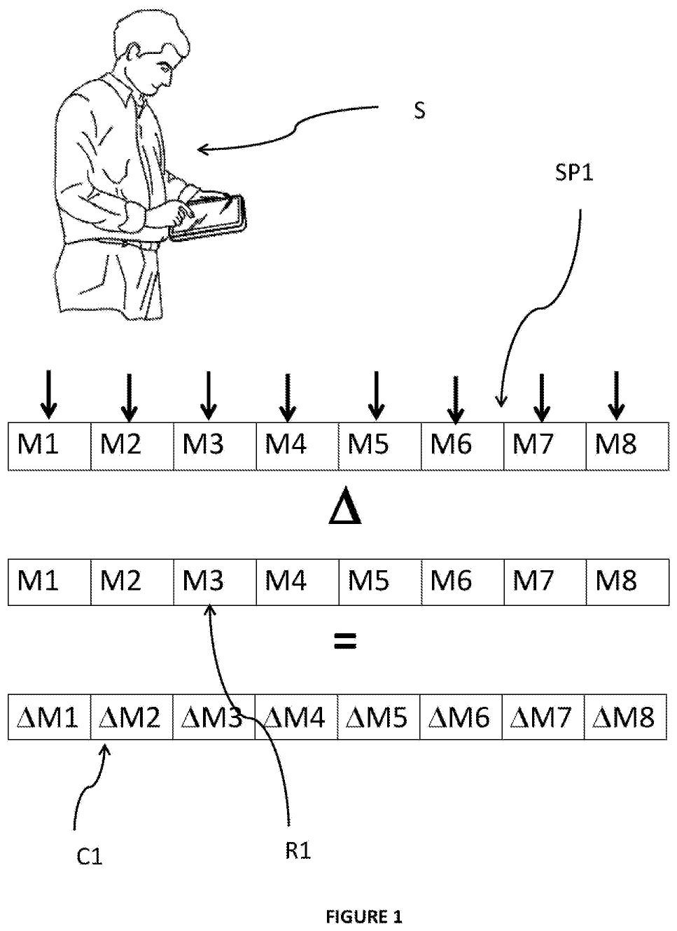

Referring to FIG. 1 a computer implemented method of determining athletic aptitude may comprise determining a plurality of metrics in relation to a sportsperson, entering the plurality of metrics into a database, and comparing the metrics with reference metrics to provide a sportsperson comparison output.

A plurality of metrics in relation to a sportsperson S are entered into a database and collected as actual values M1, M2 . . . M8 forming part of a sportsperson profile. Clearly there can be any number of metrics. A sportsperson profile may include information other than metrics, such as, for example, name, sport, team, interests etc. The database may have a reference collection of metrics forming R1 and may compare each metric value in a sportsperson profile SP1 to the reference collection of metrics R1. An output of compared collection of metrics form C1. This can be in the form of ΔM1 to ΔMB or a ratio or ranking or other comparison means.

The reference collection of metrics R1 may be a plurality of the highest metric record value stored in the database. In such an example each metric value in sportsperson profile SP1 is compared with the reference collection of the highest metric record value stored in the database. From there, each of metrics M1 through M8 are assigned a percentage and the method may involve this for every metric forming part of a testing criteria. Continuing the AFL example, that is ten (10) separate tests (as listed above). From there, the totals of the percentages may give the sportsperson an ability score and system rank. This data may then be presented on a sportsperson's profile on a graphic user interface interconnected to the database, which may take the form of a page viewable in a browser and/or the form of a table.

### B) Verify

Metric values that are equal to and/or exceed 90% of the highest recorded metric score, may result in a system notification prompting the user to review that metric explaining the ramifications of a false value. Should they confirm the metric value to be correct and accurate, a further system notification may be sent to an administrator, informing them of such a result. It will then be up to the administrator to validate the score by manual means. i.e. call the club, view or retest the player etc.

In a preferred embodiment, there is an option for users to verify their metrics. The sportsperson performs the relevant tests in the presence of an authorised tester who determines and collates corresponding verified metrics. Metrics that are verified may be indicated as such on the graphic use interface with a notation and/or vice versa in relation to unverified metrics. For example a verified metric may be indicated as such by the inclusion of a tick graphic adjacent the verified metric on the graphic user interface. Similarly a sportsperson profile may be verified. In particularly preferred embodiments, a user may select that the reference profile may only be formed from verified metrics and/or verified sportsperson profile(s).

### C) Profile Compare

Profile compare can be an important aspect of comparing a package of skills. It is unlikely that a sportsperson would have maximum metrics in each and every relevant category. Sportspeople may have excellent athletic aptitude for a particular sport due to an overall good profile of metrics. It may therefore be important to record and assess profiles.

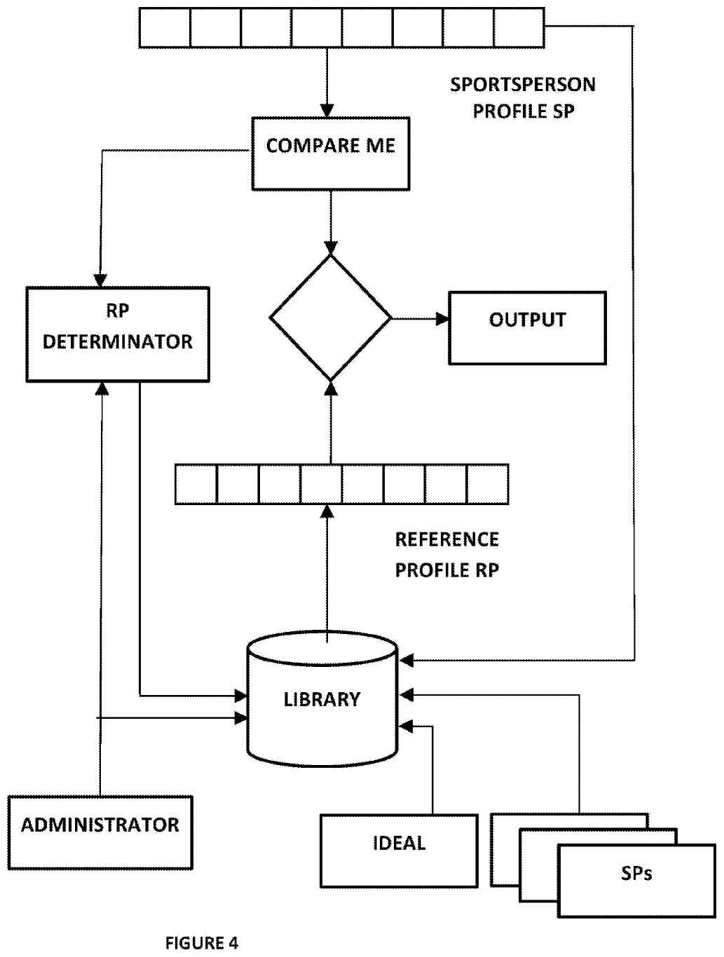

Referring to FIG. 4 there is shown a sportsperson profile SP which is entered into the database in the form of a library. The library may also include an ideal profile. The library may also include one or more other sportsperson profiles SPs. The library may also include a real time update process in which sportsperson profiles are continually entered and updated.

A computer implemented method of determining athletic aptitude may comprise determining a plurality of metrics in relation to a sportsperson, entering the plurality of metrics into a database, forming a profile of the sportsperson comprising the plurality of metrics, and comparing the sportsperson profile with a reference profile to provide a player comparison output.

The user may select one or more sportsperson profile parameters and a reference sportsperson determinator determines the reference profile to be compared to the sportsperson profile.

The reference determinator determined reference profile may include:
a) a real profile from a library of sportsperson profiles matched to the selected one or more sportsperson profile parameters.
b) a virtual profile from a library of sportsperson profiles matched to the selected one or more sportsperson profile parameters; or
c) a historical profile from the library of sportsperson profiles matched to the selected one or more sportsperson profile parameters.

The user may input the type of reference profile to be determined by the reference of sportsperson profiles matched to the selected one or more sportsperson profile parameters. Users are able to compare themselves to another profile being a Reference Profile RP by selecting, for example, a button labelled “Compare Me” on a graphic user interface, which will result in the Compare Me function output being presented on the graphic user interface.

The Compare Me function may allow users to be able to view their own sportsperson profile as well as the sportsperson profile of other users.

The reference profile may be formed from:
a) an earlier set of metrics of the sportsperson.
b) a set of metrics of a sportsperson from a library of profiles.
c) a set of metrics of one or more other sportspersons.
d) a set of metrics of a virtual sportsperson.
e) a set of best metrics to form a virtual best sportsperson.

Further the forming of the reference profile can be at real time from receipt of a plurality of other sportspersons over a network or from historical records in the library or by artificially created profiles created from the library reference profiles.

A sportsperson profile may contain all the information a user inputs into the database, as well as generated information such as:
a) Ability score
b) Rank
c) The number of Shortlists they're on
d) A line graph of their Ability score (and each of their metrics) since the date they joined the system.

An RP Determinator may also be activated by the Compare Me input of the user. The RP Determinator may determine and locate a Reference Profile RP from the Library in accordance with input details.

The Administrator function may allow control and organization of the data in the library according to a range of options and provide all of those options or a sub-set of those options to the RP Determinator for use in achieving the desired comparison to the required Reference Profile.

Sportsperson Profile pages may offer the user various system tools to automatically review and analyse metric scores and ability, as well as very quickly compare themselves to other users and averages/leading scores.

Users may be able to use a range of provided online tools to manipulate it and visually digest it. For instance, users may be able to:
i. Compare their profile, ability and metrics to that of other users or against system ambassadors (who are considered professional in that selected sport).
ii. Visually see (by use of a generated line graph displayed on a graphic user interface) how their ability (and any other specific metric) has changed over time (as well as the time they wish to analyse).
iii. Visually see how their ability or specific metric score relates to that of another user.
iv. Visually see how their ability or specific metric score relates to the average and lead score (for example, the best recorded metric score overall).
v. Generate an automated system report, that details the differences between their ability and another user and what training may be required to improve.

The prospective sportsperson list may allow the prospective sportsperson to identify or gain insight as to their potential and/or better know their competition. This list may essentially take the form of an output table. It may present all users in the form of a table, whose information can be sorted and filtered by users.

By default, the prospective sportsperson list may be sorted by Ability (from Highest to Lowest) and contain one or more of the following columns:
Ability score
Name
Age
Position
State

Users may have access to this list and may therefore be able to sort/filter it by sportsperson profile parameters such as, for example, by one or more of the following options:
Ability score (or any other metric recorded on the system)
Name
Age
Position
State
Everyone
Shortlisted members only (followed or following users)
Members of the same age group

From here, users may be able to view other sportsperson profiles and then compare themselves by uses of the provided system tools and the Compare Me (function).

A Compare Me function may be present on a sportsperson profile which would allow a user, viewing a profile other than their own, to quickly and simply compare themselves to that user.

In summary, the Compare Me function may automatically generate a report page that automatically illustrates, highlights and reports on the differences between the users (for example, their ability and individual metrics).

The comparative output of a sportsperson profile to a reference profile that may be provided by the Compare Me function includes:
a) the compared sportsperson profile to reference profile provides sportsperson comparison output determining which metrics need improving.
b) the compared sportsperson profile to reference profile provides sportsperson comparison output with weighting according to the sportsperson profile parameters determining which metrics more importantly need improving according to the sportsperson profile parameters of the sportsperson.
c) the compared sportsperson profile to reference profile provides sportsperson comparison output with total profile comparison analysis.
d) the compared sportsperson profile to reference profile provides sportsperson comparison output with total profile comparison analysis by weighting of metrics which are more important according to the sportsperson profile parameters of the sportsperson.
e) the compared sportsperson profile to reference profile provides sportsperson comparison output with total profile comparison analysis by comparative proportion of sportsperson concordance with reference profile.
f) the compared sportsperson profile to reference profile provides sportsperson comparison output with total profile comparison analysis by ranking of sportsperson in line with reference profiles according to the sportsperson profile parameters of the sportsperson.

The output may contain information such as:
An automated (and overlayed) line graph which illustrates both users abilities (and individual metrics) over a period of time (as well as the time they wish to analyse).
Several bar charts which illustrate both user's abilities (and individual metrics) as well as comparing them to the system average and leader (ie. The system's highest recorded score).
An automated report detailing the differences between user abilities and change depending on the metrics being analysed.

A Challenge function may be present on a sportsperson profile which would allow a user, viewing a profile other than their own, to ‘challenge’ that user in relation to one or more metrics. The challenge might require each user to re-perform the test relating to the metric(s) in a given time, and the best metrics) wins the challenge. The challenge may request/require the test to be re-performed with an authorised tester to verify the metric. Such a Challenge function may create friendly competition and generally help to improve motivation and enjoyment. Each attempt at re-performing the test may be recorded/videotaped and uploaded to the system so users can compare the videos of the each user, preferably side-by-side.

In preferred embodiments, users may also follow one another's sportsperson profiles which would allow them to receive, for example, updates as to newly entered metrics, the issuing and results of challenges, public and/or direct messaging to enable interactivity between users. It is particularly preferred that a user's sportsperson profile may be associated with one or more of the user's other social media accounts, for example, enabling updates in relation to the user's sportsperson profile (new metrics, challenges etc) to be posted to the user's social media account(s).

Real world examples of the use of the computer implemented method may assist sportspeople in the follow ways.
a) Changing positions—Users may gain insight as to where their ability sits within their desired sport and can therefore better understand what they need to work on in order to take it to the next level. This may include changing playing positions to one better suited to their current level of ability or potential.
b) Changing sports/codes—Carlin Isles—Ranked as the 36th fastest sprinter in the USA at the time, in 2012 Isles was recruited by the US National Rugby team and debuted in their sevens team against New Zealand. He scored a try in his first minute on the pitch. Use of the present invention may reduce the ‘luck’ involved in identifying sportspeople with athletic aptitude suitable for a change in sport/code.
c) Virtual competing—Users may be able to compare their abilities to other users around the world in virtual time. With that said, users from different countries aspiring to a sport that has a global talent pool can compare their abilities to see what they need to work on in order to improve and make it as a professional in that sport. In preferred embodiments, users may also interact directly forming relationships that may assist in pursuing professional sporting opportunities.
d) ROI on Personal/Athletic Development—Commitment, time and money is just the beginning of what it takes to become a professional sportsperson. For example, club registration fees, equipment, training, personal training are all part and parcel of reaching a potential and quite quickly, any or all of these requirements can start to add up.
   How much commitment, time and or money an individual sportsperson then applies to their ability is then relative to that sport and the individual and subjective to their level of ability and potential. The invention may therefore provide individual sportspeople with additional information to help them make a decisions based around the investment they give their ability and potential.

A range of hierarchical structures and sub-structures may be provided depending on creation of an account or subscription. In one aspect, an account allows a user to input metric data such as age, height, weight, time over a pre-determined distance, sport, position and the like.

Once data is inserted into various provided fields, an account member may be able to access a range of information including information from a library containing comparative information, and a relative ranking with other people. One user may compete with another user, and updated information may allow adjustment of ranking. This may promote improvements in performance and/or motivation due to the creation of competition for a relative ranking. Comparative metrics may be provided, and a report established based on the comparison of metrics. The rankings and reports may be used to help identify a sportsperson's athletic aptitude for a particular sport based on a comparison with known data, or it can identify an attribute for targeting recruitment of a sportsperson for a particular sport.

As can be seen from the foregoing description of the preferred embodiments, it is plain that the present invention may incorporate one or more of the following advantages:
Allows sportspeople/coaches/sporting administrators to identify possible changes in playing positions to one that better suits a sportsperson's current level of athletic aptitude and/or potential as well as changing sporting codes altogether.
Provides prospective professional sportspeople with a realistic understanding of what is required in their chosen sport and should they find themselves of an age where tough decisions need to be made regarding commitment to the sport or training, they can clearly decide what direction to take. For example, deciding to focus more on academics or work as they may not make it as a professional.
Allows sporting clubs/coaches to easily and accurately measure and manage individual's ability and potential.
Provide a better understanding of a sportsperson's strengths, weaknesses, playing position etc.
Provide a better understanding of the athletic aptitude of peers and prospective competition.
Allow sponsors to target a specific audience for marketing purposes.

Sporting organizations (for example, an institute of sport program such as the Australian Institute of Sport) may use the invention for rapid and accurate talent identification and recruiting for both specific and non-specific sports. As mentioned above, a sportsperson's desired sport may not necessarily be the one they're best suited to. The invention enables the collection and analysis of data to make such recommendations.

Schools and universities may also use the invention for rapid and accurate talent identification and recruiting for both specific and non-specific sports, particularly in relation to sport related scholarships.

Sporting Recruitment, Talent Agents, Scouts etc. may also use the invention for rapid and accurate talent identification and recruiting for both specific and non-specific sports.

### Interpretation

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Further, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Description of Preferred Embodiments are hereby expressly incorporated into this Description of Preferred Embodiments, with each claim standing on its own as a separate embodiment of this invention.

Further, while some embodiments described in this specification include some but not other features included in other embodiments, combinations of features of different embodiments are meant to be within the scope of the invention, and form different embodiments, as would be understood by those in the art.

### Different Instances of Objects

Unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In this specification, numerous specific details are set forth. However, it is to be understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology may be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

### Comprising

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integers or steps.

### References to Known Matter

Reference in this specification to any prior publication (or information derived from it), or to any matter which is known is not, and should not be taken as an acknowledgment or admission or any form of suggestion that that prior publication (or information derived from it) or known matter forms part of the common general knowledge in the field of endeavour to which this specification relates.

### Scope of Invention

Although preferred forms of the present invention have been described with particular reference to applications in relation to Australian Rules Football, it will be apparent to persons skilled in the art that modifications can be made to the preferred embodiments described above or that the invention can be embodied in other forms and used in alternative applications. For example, any formulas given above are merely representative of procedures that may be used. Functionality may be added or deleted from the block diagrams and operations may be interchanged among functional blocks. Steps may be added or deleted to methods described within the scope of the present invention.

### INDUSTRIAL APPLICABILITY

It should be apparent from this specification, that the arrangements described are applicable at least to the sports and fitness industries.

1-18. (canceled)

19. A computer implemented method of determining athletic aptitude, comprising:
(a) determining a plurality of metrics in relation to a sportsperson;
(b) entering the plurality of metrics into a database;
(c) forming a profile of the sportsperson comprising the plurality of metrics; and
(d) comparing the sportsperson profile with a reference profile to provide a sportsperson comparison output, wherein the reference profile is formed from a set of metrics taken from two or more other sportspersons included in the database;
wherein the sportsperson comparison output is depicted on a graphic user interface interconnected to the database,
wherein the reference profile further comprises two or more sportsperson profile parameters; and
wherein a user can select the sportsperson profile parameters via the graphic user interface and a reference determinator determines the reference profile to be compared to the sportsperson profile.

20. The computer implemented method according to claim 19, wherein the plurality of metrics relates to a team sport and the reference profile relates to a position or a sub-set of positions in the team sport.

21. The computer implemented method according to claim 19, wherein the plurality of metrics includes two or more of:
(a) Vertical Jump;
(b) Running Vertical Jump;
(c) Agility Run;
(d) 20 metre Sprint;
(e) Repeat Sprint;
(f) Shuttle Run/Beep Test;
(g) 3 kilometre Time Trial (running);
(h) Kicking Efficiency;
(i) Clean Hands;
(j) Goal Kicking Test.

22. The computer implemented method according to claim 21, wherein the plurality of metrics relates to Australian rules football.

23. The computer implemented method according to claim 19, further comprising forming the reference profile from a previously determined set of metrics in relation to the sportsperson.

24. The computer implemented method according to claim 19, further comprising forming the reference profile from another sportsperson profile included in the database.

25. The computer implemented method according to claim 19, wherein the reference profile is formed in real-time as the metrics are received over a network by the database and the other sportsperson profile(s) are formed.

26. The computer implemented method according to claim 19, further comprising forming the reference profile from a set of pre-determined metrics forming a virtual best sportsperson.

27. The computer implemented method according to claim 19, wherein one of the sportsperson profile parameter(s) is age, weight or height.

28. The computer implemented method according to claim 19, wherein one of the sportsperson profile parameter(s) is a particular sport.

29. The computer implemented method according to claim 28, wherein a further sportsperson profile parameter is a position or sub-set of positions played in the particular sport.

30. The computer implemented method according to claim 28, wherein the particular sport is Australian rules football or a rugby code.

31. The computer implemented method according to claim 19, wherein one of the sportsperson profile parameter(s) is a team of the sportsperson in the sportsperson profile.

32. The computer implemented method according to claim 19, wherein the reference determinator determines the reference profile to be from another sportsperson profile included in the database matched to the selected one or more player profile parameters.

33. The computer implemented method according to claim 19, wherein the reference determinator determines the reference profile to be formed from a set of metrics taken from two or more other sportsperson profiles included in the database matched to the selected one or more player profile parameters.

34. A computer implemented method of determining athletic aptitude, comprising:
(a) determining a plurality of metrics in relation to a sportsperson;
(b) entering the plurality of metrics into a database;
(c) forming a profile of the sportsperson comprising the plurality of metrics; and
(d) comparing the sportsperson profile with a reference profile to provide a sportsperson comparison output, wherein the reference profile is formed from a set of metrics taken from two or more other sportspersons included in the database;
wherein the sportsperson comparison output is depicted on a graphic user interface interconnected to the database, and
wherein the steps of determining a plurality of metrics in relation to a sportsperson and entering the plurality of metrics into a database is limited to numerical metrics, said metrics being selected from:
(a) Vertical Jump;
(b) Running Vertical Jump;
(c) Agility Run;
(d) 20 metre Sprint;
(e) Repeat Sprint;
(f) Shuttle Run/Beep Test;
(g) 3 kilometre Time Trial (running);
(h) Kicking Efficiency;
(i) Clean Hands;
(j) Goal Kicking Test.

35. A computer implemented method of determining athletic aptitude according to claim 34, wherein the steps of determining a plurality of metrics in relation to a sportsperson and entering the plurality of metrics into a database excludes the use of visual recordings of the sportsperson.
